(19) United States
(12) Patent Application Publication
(10) Pub. No.: US 2002/0192645 A1
(43) Pub. Date: Dec. 19, 2002

(54) BCR-ABL gene rearrangement assay method

(76) Inventors: Richard W. Tseng, (US); Michael K. Samoszuk, Rancho Santa Margarita, CA (US)

Correspondence Address: Richard J. Warburg, Foley & Lardner, 23rd Floor, 402 West Broadway, San Diego, CA, 92101-3542, US

(21) Appl. No.: 09/747,165

(22) Filed: Dec. 22, 2000

Related U.S. Application Data
(60) Provisional application No. 60/173,050, filed on Dec. 24, 1999.

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

Methods for assaying biological samples for bcr-abl translocation rearrangements and reporting the results of such assays which are useful in the diagnosis of CML and/or ALL are provided.

This application claims priority under 35 USC Section 119(e) from U.S. Provisional Patent Application Serial No. 60/173,050 filed Dec. 24, 1999. The present invention relates to assay methods which allow for the specific detection, and quantitation of bcr-abl gene rearrangements.

### FIELD OF THE INVENTION

### BACKGROUND OF THE INVENTION

The Philadelphia chromosome (Ph) is a translocation between chromosome 9 and 22 t(9;22) (q34;q11) that is found in more than 90-95% of chronic myeloid leukemia (CML) about 20-25% of adult and 2-10% of childhood acute lymphoblastic leukemia (ALL). See, Rowley J D (1973) A new consistent chromosomal abnormality in chronic myelogenous leukemia identified by quinacrine fluorescence and Giemsa staining. Nature. 243: 290-293. Catovsk D (1979) Ph1 positive acute leukemia and chronic granulocytic leukemia: one or two disease. Br. J. Haematol. 42: 493-498. Prist J R, Robison L, McKenna R W, Linquist L L, Warkentin P I, LeBien T W, Woods W G, Kersey J H, Coccia P F, and Nesbit M E Jr. (1980) Philadelphia chromosome positive childhood acute lymphocytic leukemia. Blood. 56: 15-22. In CML, most of the translocation falls in the major breakpoint cluster region (M-bcr) of the BCR gene, and results in two BCR-ABL mRNA molecules with a b2a2 or b3a2 junction which encode p210^{BCR-ABL} fusion protein. See, Konopka J B, Watanabe S M, and Witte O N. (1984). An alteration of the human c-abl protein in K562 leukemia cells unmasks associated tyrosine kinase activity. Cell. 37: 1035-1042. In ALL, about two thirds of the BCR breakpoint falls in the minor breakpoint cluster region (m-bcr), and the hybrid BCR-ABL transcript contains an e1a2 junction and is translated as a p190^{BCR-ABL} fusion protein. See, Clark S S, McLaughlin J, Crist W M, Champlin R, and Witte O N. (1987). Unique forms of the abl tyrosine kinase distinguish PH-positive CML from Ph-positive ALL. Science. 235: 85-88. Because CML is a clonal disease, detection of BCR/ABL fusion transcripts should precisely reflect CML disease activity. BCR-ABL mRNA can be specifically and efficiently detected by the reverse transcription-polymerase chain reaction (RT-PCR), because this fusion gene is leukemia specific it can be used as a marker to identify residual disease after therapy. Quantitative RT-PCR detection of BCR/ABL fusion is well established in CML diagnostics, and PCR positivity is virtually diagnostic of this type of leukemia. Kawasaki E S, Clark S S, Coyne M Y, Smith S D, Champlin R, Witte O n, and McCormick F P. (1988). Diagnosis of chronic myelogenous and acute lymphocytic leukemia by detection of leukemia-specific mRNA sequences in vitro. Proc. Natl Acad. Sci. USA. 85: 5698-5702. Gibson U E M, Heid C A, and Williams P M. (1996). A novel method for real time quantitative RT-PCR. Genomic Res. 6: 995-1001.

However, the present methods of CML or ALL diagnostics are lacking in that for the qualitative assay (that is indication of presence or absence of the disease and not a quantitative number of cells present or other quantitative information provided) uses hazardous radioactive isotopes, complex hybridizations and takes about five days. Further, the present methods do not provide for a single container method of assaying for all three translocation products and providing reproducible and easily and meaningfully interpretable results.

Accordingly, it would be desirable to provide assay methods for bcr-abl translocation rearrangements that are convenient to carry out, provide highly reproducible qualitative and quantitative results and in which all three translocations may be assayed for at once in one container.

### SUMMARY OF THE INVENTION

This assay is, in a preferred embodiment, a real time quantitative RT-PCR assay using, for example, the ABI PRISM® 7700 Sequence Detection System, a thermal cycler associating a laser, a detector, and real time amplification detection software (available from, for example, PE Applied Biosystems). This technique allows the quantification of a specific target, using for example, a fluorescent labeled probe and the 5′ nuclease activity of Taq DNA polymerase during the PCR process. The copy number of target sequence (BCR-ABL) or reference sequence, such as housekeeping gene GAPDH (glyceraldehyde-3-phosphate dehydrogenase), or another universally expressed gene, in a sample was calculated using a standard curve (serial dilution of K562 total RNA). K562 is a human chronic myelogenous leukemia cell line that has BCR-ABL b3a2 translocation. Specific primers and probes were designed for all 3 translocations (e1a2, b2a2 and b3a2). To distinguish these 3 translocations, the PCR product can be run on a 4% agarose gel. Based on the size of PCR fragments, the types of translocation can be determined.

In one aspect, the presently claimed subject matter is directed to a method for determining bcr-abl translocation rearrangements in a biological sample comprising the steps of:

a) extracting RNA from a biological sample;

b) quantifying the extracted RNA;

c) reverse transcribing the RNA to cDNA;

d) amplifying the cDNA and detecting a cDNA signal by using the primers and probes set forth in SEQ. ID. NOS. 1-8;

e) obtaining a standard curve of cDNA signals from serial dilutions of a leukemic cell line, wherein the cDNA is obtained by repeating steps a)-d) with the RNA from the leukemic cell line and not sample; and

f) extrapolating a measurement of the leukemic cells present in the sample by comparing the signal from step d) with that from step e).

In one aspect of the method set forth above, the amplification and detection of the cDNA in step d) is accomplished by Real Time PCR.

In another aspect of the method set forth above, the amplification and detection of the cDNA in step e) is accomplished by Real Time PCR.

Other features and advantages of the invention will become apparent from the following detailed description.

### DESCRIPTION OF THE FIGURES

FIG. 1 is a standard curve created using six duplicates FAMA1-A2, etc. of standard dilutions of leukemia cells resulting in six curves. The point where each curve crosses the threshold line (see arrow) indicates the Cycle threshold or CsubT for each sample. Theory predicts that the difference between each serial dilution curve should be 3.5. Comparison of FAM A1/A2 with FAMA3/A4 shows this result to fairly good accuracy (21.150−17.828=3.32). Also, the reproducibility between samples A1 (17.828) and A2 (17.615; A3 and A4 etc. is shown.

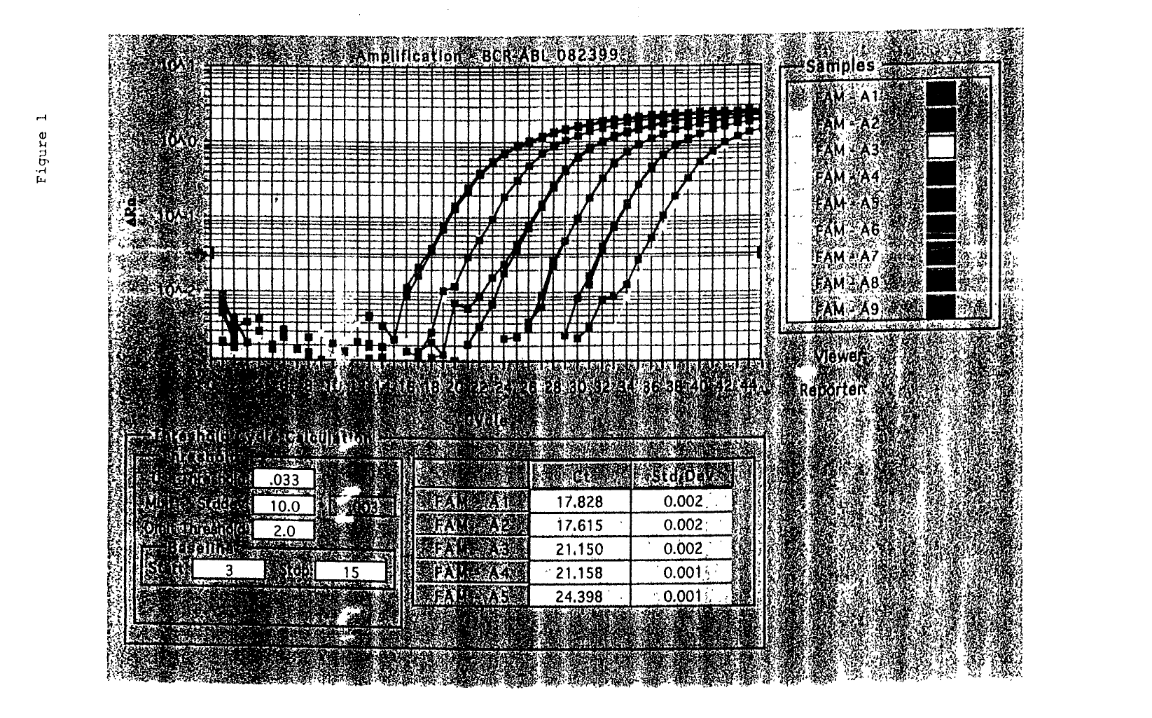

FIG. 2 is a standard curve created from the curve in FIG. 1 which allows for interpretation of the quantity of leukemic cells in an unknown sample.

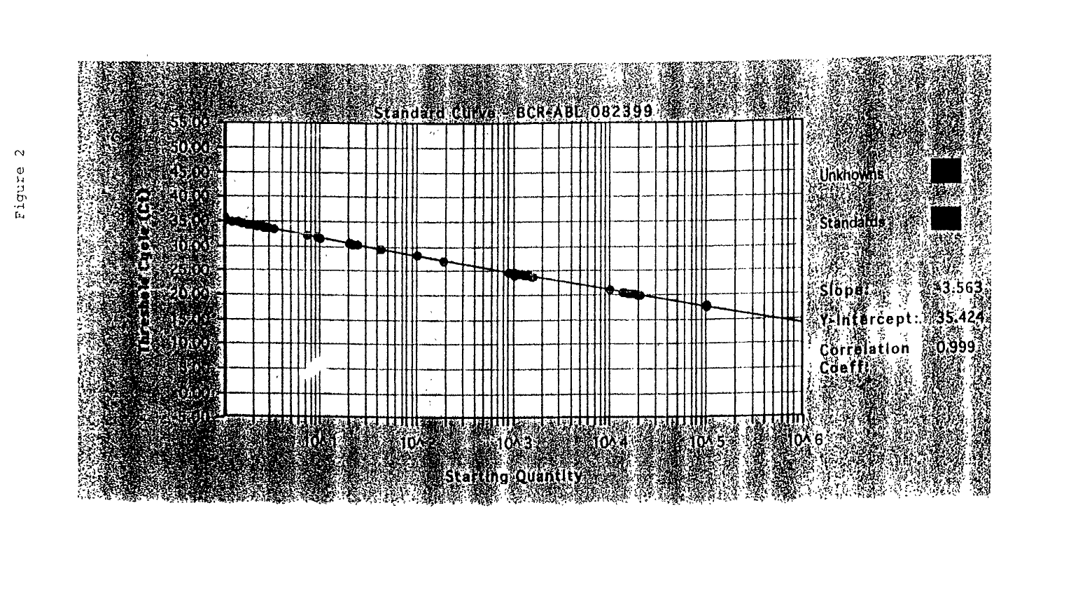

FIG. 3 illustrates an agarose gel in which the top lane M are size markers, the lane below it SUPB 15 illustrates the band position of the e1a2 PCR product K562 illustrates the band position of b3a2 and BC9-515 illustrates the band position of b2a2. The remaining lanes with BC9-516, 519, 518, 517, 274, 273 and 272 are samples which had unknown PCR bcr-abl gene translocation products.

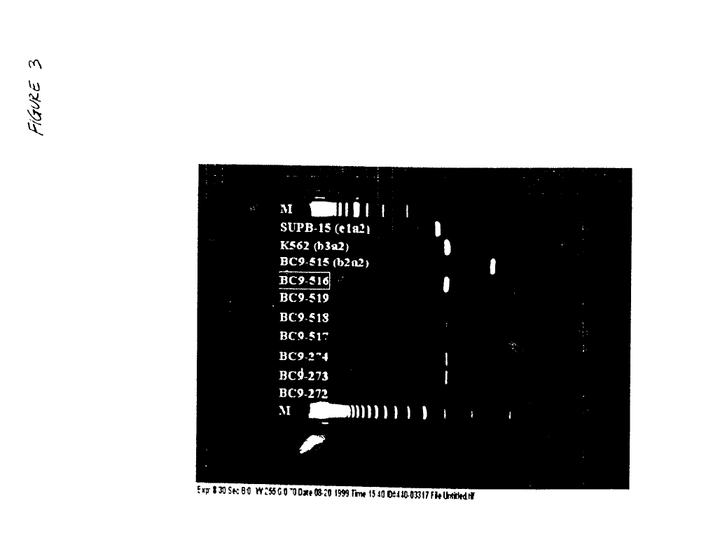

### DETAILED DESCRIPTION OF THE INVENTION

The biological samples used in this invention may be blood, bone marrow or other tissues in which CML or ALL cells may reside. An example of a sample suitable for the present invention is as follows: For assay, transfer 2 ml of peripheral blood drawn in EDTA or heparin anti-coagulant to 6 ml of stabilizing agent RNA STAT 50™ LS or TRIzol® LS (or other RNAse inhibitor), or 0.2 ml of bone marrow aspirate to 0.6 ml of stabilizing agent RNA STAT 50™ LS or TRIzol® LS (or other RNAse inhibitor). Freeze the samples and ship them frozen on dry ice.

Examples of instruments and equipment which may be used to perform the assays claimed are as follows (it being understood that equivalents as would be known to those of ordinary skill in the art could also be used)

ABI PRISM® 7700 Sequence Detection System (PE Applied Biosystems).

Biological Safety Cabinet (Baker model SGII-600 Sterilgard, or equivalent). BioLumin 960™ fluorescent plate reader configured with a 485 nm (10 nm bandwidth) excitation filter and a 520 nm (10 nm bandwidth) emission filter (Molecular Dynamics, Sunnyvale, Calif.)

Beckman Centrifuge with Swing-bucket Rotor.

Digital Imaging & Analysis System (Alpha Innotech Corporation, or equivalent).

DNA Cleanbox Workstation (CBS Scientific, Cat. # CB-120, or equivalent).

DNA Speed Vac (Savant, DNA 110).

DNA Thermal Cycler 480 (Perkin Elmer).

Eppendorf Multipurpose Centrifuge Model 5810R (Brinkmann).

Eppendorf Vacufuge™ (Brinkmann).

Freezer −20° C. and −70° C.

Horizontal Gel Electrophoresis Units.

Hot Plate/Stirrer (Corning, Cat. #6795-320, or equivalent).

Microcentrifuge Non-refrigerated, Eppendorf Model 5417C (Brinkmann).

Microcentrifuge Refrigerated, Eppendorf Model 5417R (Brinkmann).

Microwave Oven.

MJ PTC-200 DNA Engine Thermal Cycler (MJ Research).

Pipet-Aid, Drummond (VWR, Cat. #53498-001, or 53498-056).

Pipettors:

P-10 (Ranin, Cat. #P-10, or equivalent).

P-20 (Ranin, Cat. #P-20, or equivalent).

P-200 (Ranin, Cat. #P-200, or equivalent).

P-1000 (Ranin, Cat. #P-1000, or equivalent).

Power Cell for Gel Electrophoresis.

Refrigerator.

UV Transilluminator with Protective Cover (International Biotechnologies, Inc.).

Vortexer.

Examples of reagents and supplies which may be used to perform the assays claimed are as follows (it being understood that equivalents as would be known to those of ordinary skill in the art could also be used, unless otherwise indicated)

Barrier Pipet Tips: 10 μl, 20 μl, 200 μl, and 1000 μl.

Beakers: 100 ml, 250 ml, 600 ml, and 1000 ml.

Centrifuge Tubes, Disposable, Polypropylene, Sterile 15 and 50 ml.

Ehrlenmeyer Flasks: 100 ml, 250 ml, and 1000 ml.

Graduated Cylinders: 100 ml, 500 ml, and 1000 ml.

Microcentrifuge tubes 2.0 ml Screw Cap with O-ring, Sterile (Sarstedt, Cat. # 72693005, or equivalent).

Microcentrifuge tubes 1.5 ml Screw Cap with O-ring, Sterile (Sarstedt, Cat. # 72692005, or equivalent).

Microcentrifuge tubes 1.7 ml (National Scientific, Cat. # CN065S-GT, or equivalent).

Microcentrifuge tubes 0.65 ml (National Scientific, Cat. # CN170S-GT, or equivalent).

MicroAmp® 96-Well Bases (PE Applied Biosystems, Cat. # N801-0531, or equivalent).

MicroAmp® Optical 96-well Reaction Plate and Optical Caps (PE Applied Biosystems, Cat. # 403012, or equivalent).

MicroAmp® Optical Caps (8 Caps/Strip) (PE Applied Biosystems, Cat. # N801-0935, or equivalent).

NuSieve® 3:1 Agarose (FMC, Cat. # 50090, or equivalent).

Pipet Tips 20 μl, 200 μl, and 1000 μl.

Polypropylene Conical Tubes 15 ml and 50 ml.

Sterile Serological Pipets 5 ml, 10 ml and 25 ml.

Stirring Bars.

Transfer Pipettes (Sterile, Fine Tip) (Samco Scientific Inc., Cat. # 232-20S, or equivalent).

Transfer Pipettes, B/B-PET (Samco Scientific Inc., Cat. # 336, or equivalent).

Amplitaq Gold™ DNA Polymerase (PE Applied Biosystems, Cat. # N808-0243, or equivalent).

Bromphenol Blue (Sigma, Cat. # B-6131, or equivalent).

Cell Line K562 Human Chronic Myelogenous Leukemia (ATCC, Cat. # CCL-243).

Cell Line SUP B15 Human B Cell Acute Lymphoblastic Leukemia (ATCC, Cat. # CRL-1929).

Chloroform, Biotechnology Grade (Amresco Cat. # 0757, or equivalent).

dNTP Set, PCR Grade, 100 mM each of dATP, dCTP, dGTP, dTTP (Roche Molecular Biochemicals, Cat. # 1 969 064, or equivalent).

Distilled Water, DNase/RNase-Free, (ICN Cat. # 821739, or equivalent).

DMSO (Dimethyl Sulfoxide) (Amresco, Cat. # 0231, or equivalent).

DNA Ladder 100 bp (Gibco/BRL, Cat. # 15628-050, or equivalent).

0.5 M EDTA, pH 8.0, RNase Free (Ambion, Cat. # 9260G, or equivalent).

Ethidium Bromide Tablet (100 mg/Tablet) (Sigma, Cat. # E-2515, or equivalent).

Ethyl Alcohol (Ethanol), 200 proof, Dehydrated Alcohol, U.S.P. Punctilious (Quantum Chemical Co, or equivalent).

Ficoll (Sigma, Cat. # F-2637, or equivalent).

GeneAmp® 10× PCR Buffer II & 15 mM MgCl₂ Solution (100 mM Tris-HCl pH 8.3, 500 mM KCl) (PE Applied Biosystems, Cat # N808-0130, or equivalent).

Isopropyl Alcohol, Biotechnology Grade (Amresco Cat. #0918, or equivalent).

Random Primers (Roche Molecular Biochemicals, Cat. # 1 034 731, or equivalent).

RiboGreen™ RNA Quantitation Kit (Molecular Probes, Cat. # R-11490) Applicants have found that this dye seems to work particularly well for the claimed method.

The kit including: RiboGreen reagent (1 ml), Ribosomal RNA standard, 20× TE buffer (pH 7.5) RNase-free.

RNaseZap (Ambion, Cat. # 9780, or equivalent).

RNasin Ribonuclease Inhibitor (Promega, Cat. # N2515, or equivalent).

RNA STAT 50 LS™ (TEL-TEST Inc, Cat. # CS-115, or equivalent).

Sodium Azide (VWR, Cat. # JTV015-5, or equivalent).

Sodium Chloride (Amresco, Cat. # 0241, or equivalent).

5M NaCl, RNase Free (Ambion, Cat. # 9760G, or equivalent).

Sodium Phosphate, Dibasic (Amresco, Cat. # 0348, or equivalent).

SuperScript II RNase H⁻Reverse Transcriptase (Supplied with a vial of 5× first strand buffer and a vial of 100 mM DTT) (Life Technologies, Cat. # 18064-014, or equivalent).

10× TBE (Tris-Borate-EDTA) Buffer (Amresco, Cat. # 0658, or equivalent).

1 M Tris, pH 7.0, RNase Free (Ambion, Cat. # 9850G, or equivalent).

1 M Tris, pH 8.0, (Amresco, Cat. # E199, or equivalent).

TRizol® LS (Life Technologies, Cat. # 10296, or equivalent).

Xylene Cyanol FF (Kodak, Cat. # IB72120, or equivalent).

6-ROX, SE (6-carboxy-X-rhodamine, succinimidyl ester) (Molecular Probes, Cat. #C-6126, or equivalent).

25 mM dNTPs' Solution (for 1.0 ml)

(25 mM of dATP, dCTP, dGTP, and dTTP each)

Mix 250 μl each of 100 mM dATP, dCTP, dGTP, dTTP.

Aliquot 100 μl per 0.65 ml microtubes.

Stable at −20° C. for 1 year.

100 bp DNA Ladder Working Stock Solution (0.07 μg/μl)

For 1000 μl

| | | | |
|---|---|---|---|
| Take | 70 | μl (70 μg) | 100 bp DNA ladder |
| Add | 760 | μl | Molecular biology grade water |
| Add | 170 | μl | 6X gel dye |

Store at −20° C. Stable for one year.

75% Ethanol Working Solution

In a sterile 50 ml conical tube, add 37.5 ml of 200 proof ethanol to 12.5 ml DNase/RNase free distilled water.

Stable at 20-30° C. for one month. However, it is recommended that to be prepared fresh weekly.

10 mg/ml Ethidium Bromide Solution

Dissolve 1 pellet (100 mg) of ethidium bromide in 9 ml distilled water.

Bring up the final volume to 10 ml in a 15 ml conical centrifuge tube.

Place into brown bottle or foil-cover container.

Stable at 20-30° C. for 6 months.

CAUTION: Ethidium bromide is a mutagen and must be handled carefully.

6× FEBX

Formula

0.1% Bromphenol Blue (BPB), 0.1% Xylene Cyanol, 6% Ficoll, 0.1M EDTA

For 50 ml:

In a 50 ml conical centrifuge tube, add 35 ml distilled water, 0.05 g BPB, 0.05 g Xylene Cyanol, 3.0 g Ficoll, 10 ml 0.5M EDTA.

Incubate in 60° C. water bath to accelerate the dissolving of Ficoll, swirl the tube from time to time during the incubation.

Bring up the final volume to 50 ml with distilled water.

Stable at 20-30° C. for 12 months.

6× Gel Loading Dye

Formula

0.06% Bromphenol Blue (BPB), 6% Ficoll, 0.1M EDTA

| | |
|---|---|
| Bromphenol Blue | 0.03 g |
| Ficoll | 3.00 g |
| 0.5 M EDTA (pH 8) | 10 ml |

Bring up the volume to 50 mls with distilled water

Stable at 20-30° C. for 12 months.

0.3 μg/μl Random Primers Working Dilution

Mix 10 μl 3 μg/μl Random Primers

and 90 μl Distilled Water (DNase/RNase Free)

Store at −20° C. Stable for one year.

100 μM Oligo-nucleotide (primer and probe) Stocks

The formula to be followed for preparing the stock solution of primers and probes is dependent upon the amount of the oligo-nucleotide provided by the manufacturer.

There are 3 primers and 2 probes for BCR-ABL and 2 primers and 1 probe for GAPDH internal control.

Use the following formula to make your primer stock:

1,000 pmoles in 1,000 μl of distilled water=μM final concentration (pmoles/μl).

Therefore, if you have 10,000 pmoles and you want to make 100 μM stock solution.

10,000 pmoles÷100 μM=100 μl

10,000 pmoles of primer diluted into 100 μl of water=100 μM primer concentration.

Take the yield amount from the manufacturer, e.g. ABL-R is 24997 pmoles

24,997 pmoles÷100 μM=249.97 μl=250 μl

Dissolve the pellet in 250 μl of water will give a final concentration of 100 μM.

Store all oligonucleotide stock solutions at −20° C. and stable for at least 1 year.

25 μM Primer and Probe Working Stocks

Make 1:4 dilution in water from the 100 μM stock.

Mix 50 μl 100 μM Stock

and 150 μl Distilled Water

Store at −20° C. Stable for one year.

RNA Quantitation Standard Working Stock (2,000 ng/ml)

Take out a tube of RNA standard (RiboGreen RNA Quantitation Kit, 100 μg/ml, 200 μl) from the −70° C. freezer.

Transfer 200 μl of 100 μg/ml RNA solution to 9.8 ml of TE buffer (pH 7.5) (1:50 dilution).

Aliquot 1 ml to each 1.7 ml sterile microcentrifuge tubes (screw cap with O-ring).

Store at −70° C. and stable for 1 year.

TaqMan PCR Master Reaction Mix for BCR-ABL:

Formula:

1× PCR Buffer II [10 mM Tris-HCl (pH 8.3), 50 mM KCl]

4.0 mM MgCl₂

0.2 mM dNTPs (0.2 mM of dATP, dCTP, dGTP, and dTTP each)

0.2 μM BCR-P1F

0.2 μM BCR-P2,P3F

0.2 μM ABL-R

0.1 μM P1 Probe

0.1 μM P2,P3 Probe

0.3 μM 6-ROX

0.025 Unit/μl AmpliTaq Gold DNA Polymerase

| | 1X (μl) | 26X (μl) | 50X (μl) |
|---|---|---|---|
| 10X PCR Buffer II | 3.00 | 78.00 | 150.0 |
| 25 mM MgCl₂ | 4.80 | 124.80 | 240.0 |
| 25 mM dNTPs | 0.24 | 6.24 | 12.0 |
| 25 μM BCR-P1F | 0.24 | 6.24 | 12.0 |
| 25 μM BCR-P2,P3F | 0.24 | 6.24 | 12.0 |
| 25 μM ABL-R | 0.24 | 6.24 | 12.0 |
| 25 μM P1 Probe | 0.12 | 3.12 | 6.0 |
| 25 μM P2,P3 Probe | 0.12 | 3.12 | 6.0 |
| 100 μM 6-ROX | 0.09 | 2.34 | 4.5 |
| AmpliTaq Gold (5U/μl) | 0.15 | 3.90 | 7.5 |
| Distilled Water | 15.76 | 409.76 | 788.0 |
| Total | 25.00 | 650.00 | 1250.0 |

TaqMan PCR Master Reaction Mix for GAPDH Internal Control:

Formula:

1× PCR Buffer II [10 mM Tris-HCl (pH 8.3), 50 mM KCl]

4.0 mM MgCl₂

0.2 mM dNTPs (0.2 mM of dATP, dCTP, dGTP, and dTTP each)

0.2 μM GAPDH-F

0.2 μM GAPDH-R

0.1 μM GAPDH Probe

0.3 μM 6-ROX

0.025 Unit/μl AmpliTaq ® Gold DNA Polymerase

| | 1X (μl) | 26X (μl) | 50X (μl) |
|---|---|---|---|
| 10X PCR Buffer II | 3.00 | 78.00 | 150.0 |
| 25 mM MgCl₂ | 4.80 | 124.80 | 240.0 |
| 25 mM dNTPs | 0.24 | 6.24 | 12.0 |
| 25 μM GAPDH-F | 0.24 | 6.24 | 12.0 |
| 25 μM GAPDH-R | 0.24 | 6.24 | 12.0 |
| 25 μM GAPDH Probe | 0.12 | 3.12 | 6.0 |
| 100 μM 6-ROX | 0.09 | 2.34 | 4.5 |
| AmpliTaq Gold (5U/μl) | 0.15 | 3.90 | 7.5 |
| Distilled Water | 20.12 | 523.12 | 1006.0 |
| Total | 29.00 | 754.00 | 1450.0 |

1× TBE

Formula:

0.089 M Tris Base

0.089 M borate

2 mM EDTA

For 2000 ml, add 200 ml 10× TBE buffer to 1800 ml distilled water.

Mix well.

Stable at 20-30° C. for 3 months.

1× TE (Tris-EDTA) Buffer pH 7.0, RNase Free (for 5000 μl)

Formula:

10 mM Tris-HCl pH 7.0

1 mM EDTA

| | | | |
|---|---|---|---|
| Mix | 50 | μl | 1 M Tris-HCl (pH 7.0) (RNase Free), |
| | 10 | μl | 0.5 M EDTA (pH 8.0) (RNase Free), |
| | 4940 | μl | Distilled Water (DNase/RNase Free). |
| Total | 5000 | μl. | |

Aliquot 1 ml each to five 1.7 ml DNase/RNase Free microtubes (have the screw cap with O-ring).

Stable at 20-30° C. for 1 year.

1× TE (Tris-EDTA) Buffer (pH 7.5), RNase Free (for 5000 μl)

Formula:

10 mM Tris-HCl pH 7.5

1 mM EDTA

For 2000 ml, add 200 ml 10× TE buffer(pH 7.5) to 1800 ml DNase/RNase free water.

Mix well.

Stable at 20-30° C. for 6 months.

1× TE (Tris-EDTA) Buffer (pH 8.0) (for 100 ml)

Formula:

10 mM Tris-HCl (pH 8.0)

1 mM EDTA

| | | | |
|---|---|---|---|
| Mix | 1.0 | ml | 1 M Tris-HCl (pH 8.0), |
| | 0.2 | ml | 0.5 M EDTA (pH 8.0), |
| | 98.8 | ml | Distilled Water (DNase/RNase Free). |
| Total | 100 | ml. | |

Stable at 20-30° C. for 1 year.

100 μM 6-ROX Working Solution

For 5 mM 6-ROX, dissolve 5 mg (one bottle) of 6-ROX in 1 ml of DMSO.

For 100 μM 6-ROX working solution transfer 20 μl of 5 mM stock solution to 980 μl of distilled water (Dnase/RNase Free).

Aliquot 50 μl per 0.65 ml microtube.

Store at −20° C. in dark, stable for at least 1 year.

The following documents also provide guidance on the performance of the claimed method and are incorporated herein by reference: ABI Prism® 7700 Sequence Detection System User's Manual, by PE Applied Biosystems. BioLumin™ 960 User's Guide (Molecular Dynamics, Sunnyvale, Calif.). Xperiment™ User's Guide (Molecular Dynamics, Sunnyvale, Calif.). BioLumin™/Xperiment™ New Features (Molecular Dynamics, Sunnyvale, Calif.). RiboGreen™ RNA Quantification Reagent and Kit Product Information Sheet (Molecular Probes, Eugene, Oreg.). RNA STAT-50™ LS Information sheet (Tel-Test, Inc., Friendswood, Tex.). TRIzol® LS Reagent Information Sheet (Life Technologies, Grand Island.

The primers and probes of the present invention are:

| | | |
|---|---|---|
| Primers. | | |
| BCR-P1F | SEQ ID. NO.1: | 5′ CCTCGCAGAACTCGCAACA 3′ |
| BCR-P2,P3F | SEQ ID. NO.2: | 5′ GAGCTGCAGATGCTGACGAA 3′ |
| ABL-R | SEQ ID. NO.3: | 5′ TCAGACCCTGAGGCTCAAAGTG 3′ |
| GAPDH-F | SEQ ID. NO.4: | 5′ GAAGGTGAAGGTCGGAGTC 3′ |
| GAPDH-R | SEQ ID. NO.5: | 5′ GAAGATGGTGATGGGATTTC 3′ |
| Probes. | | |
| BGR-ABL P1 | SEQ ID. NO.6: | 5′ FAM-ACACGACAACCGGGCAGTGCC-TAMRA 3′ |
| BCR-ABL P2,3 | SEQ ID. NO.7: | 5′ FAM-TGCTGTGGACAGTCTGGAGTTTCACACA-TAMRA 3′ |
| GAPDH | 5′ SEQ ID. NO.8: | JOE-CAAGCTTGCGGTTCTCAGCC-TAMRA 3′ |

The primers and probes set forth above have been designed to be able to amplify and detect all three translocations of the bcr-abl gene, namely; e1a1, ba2a2 and b3a2, without interfering with each other and providing highly reproducible results. It is to be understood that one of ordinary skill in the art using the teaching of this specification could modify the primers and probes set forth above to obtain other primers and probes which would be suitable for use in the assays set forth herein.

Example of Assay Performance

7.1 Each batch of cDNA synthesis should include a “no RNA” control.

7.2 Each TaqMan PCR should include a positive, “no RNA”, and “no sample” controls.

7.3 Positive controls should include all translocations (e1a2, b2a2, and b3a2) at 1 ng level.

7.4 Positive control b3a2 is the RNA extracted from cell line K562, e1 a2 is the RNA from cell line SUP B15, and b2a2 is from b2a2 positive samples.

7.6 The internal control for quality of RNA consists of the transcript of a housekeeping gene or universally expressed gene such as GAPDH found in all cell types.

7.7 If any controls fail in the assay, the assay should be repeated.

8.2.2 Remove all the reagents needed for this assay from refrigerator and freezer, and allow them to reach room temperature.

8.2.3 Clean the working area and instruments for the assay. Turn on instruments.

8.3RNA Extraction:

8.3.1 Label one clean 15 ml sterile centrifuge tube for each blood sample, and a 1.7 ml microtube for each bone marrow sample.

8.3.2 If sample is lysed in RNA STAT 50™ LS or TRIzol® LS and shipped frozen, quickly defrost the sample by swirling in 37° C. water bath. For peripheral blood, transfer sample (8 ml) to its labeled tube using a fine bore transfer pipet. For bone marrow, transfer sample (0.8 ml) to its labeled microtube using a fine bore transfer pipet. If the sample is in EDTA or heparin anti-coagulant, add 2 ml of whole blood 6 ml of RNA STAT 50™ LS or TRIzol® LS; or 0.2 ml of bone marrow to 0.6 ml of RNA STAT 50™ LS or TRIzol® LS in a 1.7 ml microtube . Pipet solution up and down or vortex to homogenize the sample and leave at room temperature for 10 minutes.

8.3.3 Add 1.6 ml of chloroform to 8 ml of homogenate or 0.16 ml of chloroform to 0.8 ml of homogenate. Cap tube tightly, and shake vigorously for 15 seconds (Do not vortex). Repeat with next sample. Leave it at room temperature (15-30° C.) for 10 minutes.

8.3.4 Centrifuge 15 ml sample tubes at 3400 rpm in Eppendorf 5810R centrifuge at 4° C. for 30 minutes, or spin microtubes at 10,700 rpm in Eppendorf 5417R centrifuge at 4° C. for 15 minutes to separate phases.

8.3.5 During centrifugation, label a new set of tubes for each sample and transfer 4 ml of isopropanol to each 15 ml tube, and 0.4 ml of isopropanol to each microtube. Change gloves.

8.3.6 Using a new fine-bore transfer pipette, transfer the aqueous phase (^{˜}4 ml or 0.4 ml) to its new tube, and discard the lower phase.

8.3.7 Cap tubes tightly, mix completely by inverting tubes several times, and place the samples at room temperature (20-30° C.) for 10 minutes.

8.3.8 Centrifuge samples (in 15 ml tube) at 3,400 rpm in Eppendorf 5810R centrifuge at 4° C. for 15 minutes or at 14,000 rpm in Eppendorf 5417R centrifuge at 4° C. for 10 minutes. The RNA will form a precipitate at the bottom of the tube.

8.3.9 Aspirate and discard the supernatant using a new transfer pipet, and return tube to ice. Repeat with next sample.

8.3.10Label a set of 1.7 ml microtubes for each sample.

8.3.11Add 1000 μl cold 75% ethanol to each sample, gently vortex to wash the pellet, and transfer to the labeled 1.7 ml microtube. There is no need to transfer for samples are already in microtubes, just gently vortex.

8.3.12Centrifuge for 10 minutes at 14,000 rpm in a microcentrifuge at 2-4° C.

8.3.13Carefully aspirate and discard supernatant, and place tube on ice.

8.3.14Place tubes in Eppendorf Vacufuge or Speed Vac with the caps opened and pointing outward.

8.3.15Dry the RNA pellet on medium heat (45° C.), for 5 minutes to remove traces of ethanol. DO NOT OVERDRY PELLETS. The dried pellet is stable at room temperature for a few hours, but should be stored at −70° C. for extended periods, and is stable indefinitely at this temperature. If the samples are to be stored at this point, be sure to label the tubes with a working #, the date, and the designation ‘RNA’, using yellow labels.

8.4 RNA Quantification:

RNA quantification is accomplished by using RiboGreen™ RNA Quantitation Kit (Molecular Probes), a fluorescence-based assay for RNA in solution, and the fluorescence is detected by BioLumin™ 960 Instrument.

BioLumin™ 960 Instrument Configuration

Configuration and use of the BioLumin™ 960 is carried out through the Xperiment™ 1.1.0 software interface on an Apple Macintosh™ computer connected to the. The abbreviated instructions below assume familiarity with the Xperiment™ software User's Guide. Refer to the User's Guide for more detailed instructions. This configuration procedure need only be carried out once when the software is first used or if the Xperiment™ preferences file in the Macintosh™ System Folder becomes corrupted or lost.

Preparation of Calibration Standards and Unknowns

8.4.1. Bring out a tube of RNA working stock solution (2,000 ng/ml) from −70° C. freezer, and let it thaw and kept it on ice.

8.4.2 Prepare 204 μl each of five calibration standards by diluting the working solution in TE (pH 7.5):

| Standard (ng/ml) | | working solution | |
|---|---|---|---|
| (Final Concentration) | Dilution | (l) | 1x TE (l) |
| 1000 | none | 204 | 0 |
| 750 | 3:1 | 153 | 51 |
| 500 | 1:1 | 102 | 102 |
| 250 | 1:3 | 51 | 153 |
| 100 | 1:10 | 20.4 | 183.6 |

Note: the concentrations in the table above are the final RNA concentrations after the solutions are brought to a final volume of 200 I with the RiboGreen reagent. The actual concentrations above are 2× those given in the table.

8.4.3 Remove the tubes of sample RNA pellet from −70° C. freezer and leave at room temperature for few minutes.

8.4.4 Add 30 μl of RNase-free TE buffer (pH 7.0) to each tube, place them on ice and vortex few times to make sure the RNA is completely dissolved in the buffer.

8.4.5 Prepare the samples to be quantified, and record sample number on a map. The samples must be diluted in sterile 1× TE (pH 7.5) so that their final RNA concentration is within the range of the calibration standards prepared above. A 1:200 dilution in TE of samples prepared 2 ml of whole blood should be used (1.02 I RNA+202.98 I TE).

8.4.6 Prepar a working solution of RiboGreen™ reagent. Remove the Stock solution of RiboGreen™ in DMSO from −20° C. storage and thaw. Protect the tube from bright light by wrapping in aluminum foil. Prepare a working solution by diluting the stock solution 1:200 in 1×TE (e.g.: 20 I stock+3.98 ml TE would be sufficient for approx. 40 measurements). 100 I of working solution is needed for each microtiter plate well used. Wrap the tube containing the working solution in aluminum foil to protect it from light and use immediately or keep at 4° C. for 1-2 hours if necessary.

8.4.7 Transfer 100 μl of each standard (first the blank, then from low to high concentrations) (duplicates) and each unknown duplicates) to the appropriate wells of the microtiter plate(s). Add 100 μl of RiboGreen™ working solution into the wells. Shield the plate from light and let the samples equilibrate for 5 min. Note: the samples and the standard are diluted 1:2 in the wells. Thus, if the samples were prepared as 1:200 dilutions, then the sample dilution factor that needs to be entered in the Xperiment™ Plate Setup procedure is 400.

Reading the Plate on BioLumin™ 960:

8.4.8 Turn on the Apple Macintosh™ computer.

8.4.9 Go to “File” and click on “Open”.

8.4.10Under “Research and Development” file, double click on “RiboGreen” file.

8.4.11 Click open “Rbgreen Template” file. Save the file (File . . . Save as . . . ) in the folder with the current date as the filename: e.g.:RB092499.

8.4.12 Double-click on the Instrument Icon. Verify that the Mode setting is set at Fluorescence and the Dye/Probe setting is set to RiboGreen (or select RiboGreen if setting is different). Click on the Select Plate button and use the pointer to select the wells that will be used in the experiment (User's Guide, pages 3-8 to 3-9) and then click OK.

8.4.13 Double-click on the Plate Setup icon. Use the scroll bar to go to the plate(s) for the unknowns. The standards are already selected. Select the wells that you will be using and use the Unknown icon to define the wells and enter the sample dilution factors (typically 400, but see the section below on standard and sample preparation) as described in the User's Guide on pages 5-2 to 5-3 and 5-8 to 5-10.

8.4.14Double-click on the Scheduler icon and verify that the icons determining the sequence of events is set up.

8.4.15 Save the file and proceed with the experimental setup.

8.4.16 Switch on the BioLumin 960 and start the Xperiment program with the previously prepared experiment file (above). The instrument control panel should indicate “Ready” after about 2 min.

8.4.17Click on the Read icon (User's Guide pages 9-1 to 9-2) to initiate the program. Open the instrument drawer, insert the microtiter tray and close the drawer when prompted to do so by the software.

8.4.18When the instrument indicates that it has completed reading the samples, double-click on the Analysis icon and then click on the standard curve button (User's Guide pages 7-1 to 7-4). If no standard curve is displayed, click on the Data List button and select the instrument (User's Guide pages 7-3 to 7-4). Examine the standard curve. If it shows a good linear fit as estimated by visual inspection, then proceed to step 8.4.19. If the slope of the curve decreases at higher concentrations, return to the main menu, open the Preferences panel (see section above on instrument configuration) and lower the PMT voltage in increments of 10 and repeat the experimental readings until a good linear fit is obtained. Also see the guideline above under Quality Control for a data acceptance criteria.

8.4.19Click on the Fitted Data List button (User's Guide pages 7-12 to 7-15) and examine the concentration data for the samples for any results that are out of the range of the calibration standards (100-1000 ng/ml) and earmark these samples for a repeat measurement in a new experiment.

8.4.20 Use the Report Manager (User's Guide, pages 8-1 to 8-3) to print the calibration curve and the fitted data list and to be filed and stored.

8.4.21 Transfer data to Microsoft Excel spreadsheet for calculation of sample volume for cDNA synthesis.

8.4.22 Store the RNA standard and samples in −70° C. freezer.

8.5cDNA Synthesis:

8.5.1 Label an RNase-free 0.65 ml microcentrifuge tube for each sample, and label an additional tube for ‘reverse transcription mix’.

8.5.2 To the tube labeled ‘reverse transcription mix’, add the following amounts of reagent, n=number of samples+controls.

| | |
|---|---|
| 5X First Strand Buffer | 4.0 μl × (n + 2) = μl |
| 25 mM dNTPs | 1.0 μl × (n + 2) = μl |
| 0.1 M DTT | 2.0 μl × (n + 2) = μl |
| RNasin ® (40U/μl) | 1.0 μl × (n + 2) = μl |
| RT (SuperScript II, 200U/μl) | 1.0 μl × (n + 2) = μl |
| Total | 9.0 μl × (n + 2) = μl |

8.5.3 Take samples out of −70° C. freezer. Let them thaw at room temperature then keep them on ice. Vortex all samples, and centrifuge for a ‘short spin’ at 4° C. to collect all suspension at the bottom of the tube. Remove the tubes from the centrifuge and place on ice.

8.5.4 Transfer 2 μl of random primers (0.3 μg/μl) to each labeled tube.

8.5.5 Based on the RNA quantification, transfer 500 ng (x μl) of sample to the labeled microcentrifuge tube. Add y μl of RNase-free TE buffer (pH7.0), to make the total volume of 91 μl. Repeat with next sample. Should include one tube with 91 μl of RNase-free TE buffer (pH7.0) and mark as “−RNA”.

8.5.6 Cap the tube and immediately place into 70° C. Perkin Elmer Thermal Cycler 480 for 5 minutes.

8.5.7 At the conclusion of the 5 minutes at 70° C., place tubes to ice for 5 minutes.

8.5.8 Do a short spin to collect condensation to the bottom of the tube.

8.5.9 Placing the sample tubes sequentially in a rack in the cleanbox. Add 11 μl of the “reverse transcription mix” to each clinical sample tube; and mix by carefully pipetting up and down several times.

8.5.10Place tubes in Perkin Elmer Thermal Cycler 480 at 42° C. for 65 minutes (60 minutes minimum, 90 minutes maximum), then change the temperature to 95° C. for 7 minutes and cool down to 4° C. automatically.

Perkin Elmer Thermal Cycler 480 can be programmed for cDNA synthesis as following:

| | | |
|---|---|---|
| Start at | 42° C. | 65 minutes |
| Link to | 95° C. | 7 minutes |
| Link to | 4° C. | Forever |

8.5.11 Do a short spin to collect condensation to the bottom of the tube.

8.5.12Add TE buffer (pH 8) to each tube and bring the concentration to 10 ng/μl, in general for 500 ng RNA in 20 μl cDNA reaction will yield 25 ng/μl concentration, add 30 μl TE buffer will then give a concentration of 10 ng/μl.

8.5.13 Label sample tubes with the date, working #, concentrations (e.g. 10 ng/μl) and the designation ‘cDNA’ using the small red labels.

8.5.14f continuing to PCR, place samples on ice, and continue with the following PCR procedures. Otherwise, store the cDNA samples at −20° C.

8.6TaqMan Real Time PCR:

8.6.1 Thaw the stock reagents for BCR-ABL TaqMan PCR. Vortex to mix, and ‘quick spin’ to collect solutions at the bottoms of the tubes. At the same time, also thaw the cDNA of samples and Standards, and keep them on ice. Turn on the machine and Apple Macintosh™ computer 30 minutes before the assay.

8.6.2 Prepare a plate map for standards and samples set up for both BCR-ABL and GAPDH internal control PCR.

8.6.3 Label two clean tubes one with BCR and the other with GAPDH for the master mixes in a cleanbox.

8.6.4 Add the ingredients of master mix to the tube according to section 5.3.13 and 5.3.14. The amount is 1×(μl)×(N+2), N is the number of samples plus standards and controls. Vortex to mix and do a quick spin. Keep on ice.

8.6.5 Prepare Standards in the cleanbox for sample addition:

8.6.5.1 Take cDNA stock of K562 (50 ng/μl) and make first dilution of 20 ng/μl in TE pH8.0, that is transfer 20 μl of 50 ng/μl stock cDNA to 30 μl of TE (pH8). Keep the solution at −20° C. when not in use.

8.6.5.2 Make series 10-fold dilutions from 20 ng/μl down to 2 pg/μl in TE (pH8) as following: (Always make fresh dilutions for each assay.) Label four 0.65 ml microtubes as 2 ng/μl, 200 pg/μl, 20 pg/μl and 2 pg/μl.

Transfer 14.4 μl of TE (pH8) to each tube.

Transfer 1.6 μl of 20 ng/μl to 2 ng/μl tube. Vortex to mix.

Transfer 1.6 μl of 2 ng/μl to 200 pg/μl tube. Vortex to mix.

Transfer 1.6 μl of 200 pg/μl to 20 pg/μl tube. Vortex to mix.

Transfer 1.6 μl of 20 pg/μl to 2 pg/μl tube. Vortex to mix.

8.6.6 In the master mix prep cleanbox, distribute 25 μl of BCR-ABL master mix to each well of the PCR plate according to the plate map.

Discard any unused working master mix.

8.6.7 According to the plate map, distribute 29 μl of GAPDH master mix to each well of the PCR plate.

Discard any unused working master mix.

8.6.8 In sample addition cleanbox, vortex cDNA samples, and quick spin at room temperature to collect sample at the bottom of the tube.

8.6.9 For BCR-ABL, load 5 μl of each standards, sample or control into its appropriate well, and mix gently by pumping pipet.

8.6.10For GAPDH, load 1 μl of each standards, sample or control into its appropriate well, and mix gently by pumping pipet.

8.6.11 Cap the reaction plate.

8.6.12Put the plate into ABI Prism 7700.

8.6.13 Open the “Sequence Detector v1.6.3 alias” in the computer, by clicking the 7700 icon on the screen.

8.6.14Click open “BCR-ABL Template”. Save the file (File . . . Save as . . . ) in the folder with the current date as the filename: e.g.: BCR-ABL 092499.

(See ABI PRISM 7700 Sequence Detection System user's manual, chapter 3 for detail instruction of how to set up Real Time PCR.)

8.6.15Click open “Thermal Cycler Condition” to check the time and temperature as following:

| | | |
|---|---|---|
| Stage 1: | 95° C. | 10:00 minutes. |
| Stage 2: | 95° C. | 0:15 minute. |
| | 60° C. | 1:00 minute. |
| | Repeat: | 45 times. |
| Sample Volume: | | 30 μl. |

Click on “OK”.

8.6.16Click on “Show Analysis” dialog box and then click on “Run” button to start PCR.

8.6.17This PCR runs approximately 2.5 hours. Save the data at the end of PCR before doing any data analyzing.

8.7Data Analysis and Review:

8.6.1 Open the data file, e.g. BCR-ABL 092499 or continue after saving the PCR run.

8.6.2 Block all internal control wells and unused wells, click on “Sample Type” pop-up menu and select “Not In Use”.

8.6.3 Go to each sample well and enter sample ID. (High-light the well and type on the box “Sample Name”) And then click on “Save”.

8.6.4 Click on “Show Analysis” button, change to analyze menu.

8.6.5 Go to “Analysis” drop-down menu and click on “Analyze”.

8.6.6 Block all the “Standards”, go to “Analysis” drop-down menu and click on “Amplification Plot”. Move the threshold to the level that the 10⁵ pg have the Ct (threshold cycle) at around 17.72. Print the amplification plot of standard curve.

8.6.7 Go to “Standard Curve” and print the standard curve. (The correlation coefficiency should be >0.990.)

8.6.8 Go to “Window” drop-down menu, click on “Experiment Report” and print.

8.6.9 To process data for internal control, go to “File” and “Save As”, save as e.g. “BCR-ABL Cont 092499”.

8.6.10On the Background plate document, click on “Dye Layer” pop-up menu, and change the dye layer to “JOE”.

8.6.11 Click on “Sample Type” pop-up menu, and change the dye on “Standard” and “Unknown” to “JOE”.

8.6.12Block those wells of standards, click on “Sample Type” pop-up menu, and go to “Standard”.

8.6.13Assign Concentration of standards to each well from 2×10⁴ pg/μl to 2 pg/μl.

8.6.14Block all sample wells, click on “Sample Type” pop-up menu, and go to “Unknown”.

8.6.15Enter sample ID as on section 8.7.3.

8.6.16Repeat the procedure from section 8.7.4 to 8.7.8. For GAPDH, the Ct can be set by computer program.

8.6.17Save the file and transfer both files to a Jaz disk.

8.8Gel Electrophoresis:

The PCR products should be examined by gel electrophoresis when it is necessary to know the type of translocation; i.e., e1a2, b2a2 and b3a2.

The gel volume varies upon the electrophoresis unit used: 40 ml for the mini-gel unit, and 120 ml for the midi-gel unit.

The PCR Products are run on a 4% NuSieve 3:1 Agarose Gel.

8.8.1 For a 4% gel, measure out 40 mls of 1× TBE buffer in a graduated cylinder, and pour into a clean 250 ml Erlenmeyer flask. This gel volume is for CBS mini-gel unit.

8.8.2 Measure out 1.6 g of NuSieve 3:1 agarose, and add to the buffer in the flask, swirling to mix.

8.8.3 Cover the mouth of the flask with Saran-wrap, punching a few small holes in the wrap to allow steam to escape.

8.8.4 Microwave on high power for 2 minutes, watching the flask carefully, not letting it boil over.

8.8.5 Add 2 μl of 10 mg/ml ethidium bromide stock solution to the gel in the flask. Swirl to mix.

8.8.6 Leave it at room temperature for about 2 minutes to cool down a little.

8.8.7 Slowly pour the gel into the unit.

8.8.8 After the gel solidified, pour 1× TBE buffer into the unit, enough to cover the gel and add 4 μl of ethidium bromide stock solution to the reservoir of positive electrode, mix well into the buffer.

8.8.9 On gel electrophoresis area, carefully remove the lids of the tray of PCR products, and add 6 μl of 6× gel loading buffer to each tube (BCR-ABL only). Mix by pipetting.

8.8.10Load 5 μl of 100 bp ladder (0.7 82 g/μl) first and then 12 μl of the sample to each well.

8.8.11 Turn on the power supply, and run the gel at 170 volts for approximately 1.5 hour.

8.8.12After electrophoresis is complete, take a photograph of the gel using the Alpha Imager 2000 and clearly label its components. Affix photograph to the assay worksheet.

8.8.13Interpretation of Gel Electrophoresis:

| Translocation | Size of Amplified Fragment |
|---|---|
| (1) e1a2 (P1) | 219 bp |
| (2) b2a2 (P2) | 124 bp |
| (3) b3a2 (P3) | 199 bp |

e1a2 is associated with ALL, while b2a2 and b3a2 are associated with CML.

8.8.14Internal control can be run on gel to assess the quality and quantity of RNA.

8.8.15The PCR product of GAPDH is run on NuSieve 3:1 agarose gel, and size of PCR fragment is 226 bp.

8.9.2 Stability of specimens for this assay is determined by using the transcript of, for example, a house-keeping gene GAPDH as an internal amplification standard. This transcript is expressed in all cell types. The quantity of internal control will indicate: 1) the quality and quantity of RNA which is converted to cDNA, and 2) if the cDNA synthesis progressed from this RNA template is sufficient to provide adequate material for PCR amplification of BCR-ABL translocation.

8.9.3 The assay can reliably detect one K562 cell in ten milliliters of whole blood. Normally, there is 5-7×10⁶ leukocytes in one milliliter of human whole blood.

However, any units and any physical characteristics which allow for the medically or biochemically relevant comparison of different samples with respect to bcr-abl translocations and diagnosis of leukemia, in particular, CML and/or ALL are within the scope of the present invention. For example, the ratio of bcr-abl signals in, for example, fluorescence units such as threshold cycle or CsubT units to CsubT units of an internal signal from a universally expressed gene.

9. Reporting

9.1 Examination of Internal Control (GAPDH):

9.1.1 All samples should be positive.

9.1.2 Low quantity or undetectable GAPDH transcript means degradation of sample RNA; repeat RNA extraction for that sample or request a new sample.

9.1.3 For gel electrophoresis, intensity of the PCR product reflects the quantity and quality of sample RNA. All the samples should have the similar quantity of PCR product, for low quantity see section 9.1.2.

9.2 Examination of Controls:

9.2.1 Master mix no sample control

Positive:

Possible contamination in all PCR reactions.

Do not continue with the interpretation of results. Prepare fresh master mix and repeat the assay.

Negative:

Continue with analysis of positive control.

9.2.2 No RNA control (cDNA synthesis)

Positive:

Possible contamination in extracted RNA or cDNA. Do not continue with interpretation. Repeat RNA extraction or cDNA synthesis for that batch and repeat the assay.

Negative:

Continue with examination of clinical samples.

9.2.3 Positive control

| | |
|---|---|
| Positive: | Continue with analysis of negative control. |
| Negative: | Repeat assay. |
| Report | The cut off for “Positive” in this assay is 10 pg. |

9.3.1 For the quantitative assay:

Positive: Report “Positive” and “LCE”.

“LCE” is the number of chronic granulocytic leukemia cell equivalent per 10,000 normal cells”.

Report actual number extrapolated from standard curve and shown on “Experiment Report”, e.g. 10 pg=1 LCE; 254 pg=25 LCE.

Negative: Report “Negative” for those samples having a quantity of less than 10 pg as shown on the “Experiment Report”.

9.3.2 For qualitative assay:

Positive: Report “Positive” for those samples having a quantity of more than 10 pg as shown on the “Experiment Report”.

Negative: Report “Negative” for those samples having a quantity of less than 10 pg as shown on the “Experiment Report”.

9.3.3 For type of translocation when requested:

Report the type based on the size of PCR fragment as following:

| Translocation | Size of Amplified Fragment |
|---|---|
| (1) e1a2 (P1) | 219 bp |
| (2) b2a2 (P2) | 124 bp |
| (3) b3a2 (P3) | 199 bp |

Table 1 illustrates the intra-run reproducibility in K562 and SUPB-15 cell lines at various cDNA concentrations.

Table 2 illustrates illustrates the inter-run reproducibility in K562 and SUPB-15 cell lines at 10 ng of cDNA.

Table 3 illustrates that the present method can detect any one of the three bcr-abl translocations.

Table 4 illustrates that the method, i.e, the column labeled TaqMan is more sensitive than a dotblot method.

Table 5 illustrates that normal non-leukemic samples do not give a false positive signal.

The presently disclosed embodiments are to be considered in all respects as illustrative and not restrictive, the scope of the invention being indicated by the appended claims, rather than the foregoing description, and all changes which come within the meaning and range of equivalency of the claims are therefore intended to be embraced therein.

TABLE 1
BCR-ABL Intra Run Aug. 25, 1999

| | K562 (10 ng) | | SUPB-15 (10 ng) | | K562 (10 pg) | | K562 (1 pg) | |
|---|---|---|---|---|---|---|---|---|
| | Ct | Quantity | Ct | Quantity | Ct | Quantity | Ct | Quantity |
| 1 | 20.91 | 18598 | 24.88 | 1414 | 32.30 | 11.50 | 36.66 | 0.68 |
| 2 | 21.09 | 16572 | 24.81 | 1477 | 32.15 | 12.68 | 36.67 | 0.68 |
| 3 | 21.02 | 17258 | 24.83 | 1461 | 32.67 | 9.06 | 36.69 | 0.67 |
| 4 | 21.06 | 16849 | 24.82 | 1476 | 32.44 | 10.54 | 36.32 | 0.85 |
| 5 | 20.89 | 18871 | 24.81 | 1485 | 32.35 | 11.13 | 36.39 | 0.81 |
| 6 | 21.12 | 16173 | 24.87 | 1429 | 32.53 | 9.93 | 37.94 | 0.30 |
| 7 | 21.07 | 16758 | 24.79 | 1498 | 32.46 | 10.35 | 35.44 | 1.50 |
| 8 | 21.04 | 17070 | 24.7 | 1589 | 32.19 | 12.34 | 36.55 | 0.7 |
| 9 | 20.89 | 18841 | 24.73 | 1559 | 32.80 | 8.31 | 36.99 | 0.55 |
| 10 | 21.03 | 17219 | 24.89 | 1410 | 32.59 | 9.56 | 36.6 | 0.71 |
| 11 | 20.91 | 18534 | 25.05 | 1266 | 32.44 | 10.53 | 34.82 | 2.25 |
| 12 | 20.95 | 18143 | 24.83 | 1460 | 32.62 | 9.36 | 35.85 | 1.15 |
| Average | 21.00 | 17573.57 | 24.83 | 1460.17 | 32.46 | 10.44 | 36.41 | 0.91 |
| SD | 0.08 | 963.80 | 0.09 | 81.05 | 0.19 | 1.31 | 0.78 | 0.52 |
| % CV | 0.40 | 5.48 | 0.35 | 5.55 | 0.60 | 12.55 | 2.15 | 57.14 |

Reaction volume is 30 ul.

TABLE 2
BCR-ABL Inter-Run Sep. 02, 1999

| | | K562 (10 ng) | | SUPB-15 (10 ng) | | STD 10⁵ ng | |
|---|---|---|---|---|---|---|---|
| | Date | Ct | Quantity | Ct | Quantity | Ct | Threshold |
| 1 | Aug. 10, 1999 | 21.02 | 10809 | 24.22 | 1309 | 17.73 | 0.030 |
| 2 | Aug. 11, 1999 | 19.90 | 22791 | 23.63 | 2049 | 17.72 | 0.027 |
| 3 | Aug. 12, 1999 | 19.89 | 21106 | 23.61 | 1823 | 17.70 | 0.025 |
| 4 | Aug. 16, 1999 | 20.10 | 17892 | 23.70 | 1493 | 17.78 | 0.022 |
| 5 | Aug. 17, 1999 | 20.9 | 18781 | 23.96 | 1529 | 17.71 | 0.029 |
| 6 | Aug. 23, 1999 | 20.39 | 16744 | 24.39 | 1539 | 17.72 | 0.033 |
| 7 | Aug. 25, 1999 | 20.14 | 19311 | 24.19 | 1368 | 17.73 | 0.028 |
| Average | | 20.22 | 18204.84 | 23.95 | 1585.84 | 17.73 | |
| SD | | 0.18 | 1629.89 | 0.33 | 167.27 | 0.03 | |
| % CV | | 0.88 | 8.95 | 1.36 | 10.55 | 0.17 | |

TABLE 3
BCR-ABL TaqMan RT-PCR (BCR-ABL071299) P1, P2 and P3 (Threshold @ 0.020)

| | Ct | | | Quantity (pg) | | | Translocation | |
|---|---|---|---|---|---|---|---|---|
| Sample | #1 | #2 | Average | #1 | #2 | Average | TaqMan | Dot Blot |
| K562 Spk 10⁶ (0.01 ml) | 21.15 | 20.77 | 20.96 | 52613 | 65768 | 59190 | Pos | |
| K562 Spk 10⁵ (0.02 ml) | 17.34 | 17.56 | 17.45 | 515059 | 450642 | 482850 | Pos | |
| K562 Spk 10⁴ (0.02 ml) | 21.08 | 20.87 | 20.98 | 54604 | 62021 | 58312 | Pos | |
| K562 Spk 10³ (0.02 ml) | 23.64 | 23.92 | 23.78 | 11774 | 9951 | 10862 | Pos | |
| K562 Spk 10² (0.02 ml) | 28.45 | 28.28 | 28.37 | 658 | 729 | 694 | Pos | |
| K562 Spk 10 (00.2 ml) | 32.84 | 31.01 | 31.93 | 47 | 142 | 95 | Pos | |
| Normal (2.0 + 0) (0.02 ml) | 45.00 | 45.00 | 45.00 | 0 | 0 | 0 | Neg | |
| Normal (1.0 + 1.0) (0.02 ml) | 45.00 | 39.38 | 42.19 | 0 | 1 | 0 | Neg | |
| K562 Spk 10⁶ (100 ng) | 16.29 | 16.33 | 16.31 | 966643 | 942784 | 954713 | Pos | |
| K562 Spk 10⁵ (100 ng) | 17.95 | 17.77 | 17.86 | 357147 | 398169 | 377658 | Pos | |
| K562 Spk 10⁴ (100 ng) | 21.11 | 21.11 | 21.11 | 53909 | 53651 | 53780 | Pos | |
| K562 Spk 10³ (100 ng) | 23.31 | 23.14 | 23.23 | 14339 | 15884 | 15112 | Pos | |
| K562 Spk 10² (100 ng) | 28.60 | 28.70 | 28.65 | 602 | 565 | 584 | Pos | |
| K562 Spk 10 (100 ng) | 30.29 | 30.36 | 30.33 | 218 | 209 | 214 | Pos | |
| Normal (2.0 + 0) (100 ng) | 34.52 | 35.41 | 34.97 | 17 | 10 | 14 | Neg? | |
| Normal (1.0 + 1.0) (100 ng) | 45.00 | 45.00 | 45.00 | 0 | 0 | 0 | Neg | |

TABLE 4
Clinical Samples

| | | Ct | | | Quantity (pg) | | | cDNA | | | |
|---|---|---|---|---|---|---|---|---|---|---|---|
| | Sample | #1 | #2 | Ave. | #1 | #2 | Average | (ng) | TaqMan | Dot Blot | Ass Date |
| 1 | BC9-266 | 28.02 | 28.31 | 28.17 | 123.6 | 101.4 | 112.5 | 50 | Pos | P3 | Jul. 12, 1999 |
| 2 | BC9-267 | 37.94 | 40.00 | 38.97 | 0.2 | 0.0 | 0.1 | 50 | Not Detec | Neg | Jul. 12, 1999 |
| 3 | BC9-268 | 40.00 | 40.00 | 40.00 | 0.0 | 0.0 | 0.0 | 50 | Not Detec | Neg | Jul. 12, 1999 |
| 4 | BC9-269 | 20.80 | 21.28 | 21.04 | 11614.1 | 16087.6 | 13850.9 | 50 | Pos | P2, P3 | Jul. 12, 1999 |
| 5 | BC9-270* | 40.00 | 40.00 | 40.00 | 0.0 | 0.0 | 0.0 | 50 | V W Amp | Neg | Jul. 12, 1999 |
| 6 | BC9-271 | 20.71 | 20.92 | 20.82 | 17070.9 | 14843.8 | 15957.3 | 50 | Pos | P2, P3 | Jul. 12, 1999 |
| 7 | BC9-272 | 35.01 | 34.14 | 34.58 | 1.1 | 2.0 | 1.6 | 50 | Not Detec | Neg | Jul. 12, 1999 |
| 8 | BC9-273 | 40.00 | 32.86 | 36.43 | 0.0 | 4.7 | 2.4 | 50 | Not Detec | Neg | Jul. 12, 1999 |
| 9 | BC9-274 | 26.78 | 30.84 | 28.81 | 285.2 | 18.5 | 151.9 | 50 | Pos | P3 | Jul. 12, 1999 |
| 10 | BC9-514BM* | 5.00 | 45.00 | 45.00 | 0.0 | 1.1 | 0.6 | 100 | W Amp | Neg | Aug. 10, 1999 |
| 11 | BC9-515BM | 20.44 | 21.46 | 20.95 | 14131.9 | 7247.9 | 10689.9 | 100 | Pos | P2 | Aug. 10, 1999 |
| 12 | BC9-516 | 24.28 | 24.24 | 24.26 | 1128.7 | 1156.8 | 1142.8 | 100 | Pos | P3 | Aug. 10, 1999 |
| 13 | BC9-540 | 21.37 | 22.22 | 21.80 | 3836.5 | 3324.4 | 3580.5 | 100 | Pos | P1 | Aug. 11, 1999 |
| 14 | BC9-571 | 45.00 | 43.49 | 44.25 | 0.0 | 0.0 | 0.0 | 100 | Not Detec | Neg | Aug. 11, 1999 |
| 15 | BC9-572 | 45.00 | 43.01 | 44.01 | 0 | 0 | 0 | 60 | Not Detec | Neg | Aug. 12, 1999 |
| 16 | BC9-573 | 42.05 | 41.79 | 41.92 | 0 | 0 | 0 | 51 | Not Detec | Neg | Aug. 12, 1999 |
| 17 | BC9-574 | 40.54 | 45.00 | 42.77 | 0 | 0 | 0 | 50 | Not Detec | Neg | Aug. 12, 1999 |
| 18 | BC9-575A | 45.00 | 45.00 | 45.00 | 0 | 0 | 0 | 100 | Not Detec | Neg | Aug. 12, 1999 |
| 19 | BC9-576 | 45.00 | 43.75 | 44.38 | 0 | 0 | 0 | 100 | Not Detec | Neg | Aug. 12, 1999 |
| 20 | BC9-275 | 24.95 | 24.14 | 24.55 | 549.6 | 957.6 | 753.6 | 100 | Pos | P3 | Aug. 16, 1999 |
| 21 | BC9-276 | 39.28 | 36.46 | 37.87 | 0.0 | 0.2 | 0.1 | 50 | Not Detec | P2 | Aug. 16, 1999 |
| 22 | BC9-277 | 45.00 | 45.00 | 45.00 | 0.0 | 0.0 | 0.0 | 100 | Not Detec | Neg | Aug. 16, 1999 |
| 23 | BC9-278 | 45.00 | 45.00 | 45.00 | 0.0 | 0.0 | 0.0 | 100 | Not Detec | Neg | Aug. 16, 1999 |
| 24 | BC9-279 | 45.00 | 45.00 | 45.00 | 0.0 | 0.0 | 0.0 | 100 | Not Detec | Neg | Aug. 16, 1999 |
| 25 | BC9-280 | 45.00 | 45.00 | 45.00 | 0.0 | 0.0 | 0.0 | 36 | Not Detec | Neg | Aug. 16, 1999 |
| 26 | BC9-275 | 23.73 | 23.87 | 23.80 | 1222.0 | 1115.3 | 1168.6 | 50 | Pos | P3 | Aug. 17, 1999 |
| 27 | BC9-272 | 45.00 | 45.00 | 45.00 | 0.1 | 0.0 | 0.1 | 50 | Not Detec | Neg | Aug. 23, 1999 |
| 28 | BC9-273 | 45.00 | 45.00 | 45.00 | 0.1 | 0.1 | 0.1 | 50 | Not Detec | Neg | Aug. 23, 1999 |
| 29 | BC9-517 | 36.15 | 36.11 | 36.13 | 1.2 | 1.2 | 1.2 | 50 | Not Detec | Neg | Aug. 23, 1999 |
| 30 | BC9-518 | 34.88 | 35.56 | 35.22 | 1.7 | 2.7 | 2.2 | 50 | Not Detec | Neg | Aug. 23, 1999 |
| 31 | BC9-519 | 36.30 | 35.15 | 35.73 | 1.1 | 1.2 | 1.1 | 50 | Not Detec | Neg | Aug. 23, 1999 |
| 32 | BC9-520 | 35.52 | 36.03 | 35.78 | 1.8 | 1.3 | 1.5 | 50 | Not Detec | Neg | Aug. 23, 1999 |
| 33 | BC9-522 | 37.33 | 38.06 | 37.70 | 0.5 | 0.3 | 0.4 | 50 | Not Detec | Neg | Aug. 23, 1999 |
| 34 | BC9-296 | 28.76 | 29.30 | 29.03 | 114.1 | 80.5 | 97.3 | 50 | Pos | P2, P3 | Aug. 25, 1999 |
| 35 | BC9-297* | 45.00 | 45.00 | 45.00 | 13.9 | 0.0 | 6.9 | 50 | W Amp | Neg | Aug. 25, 1999 |
| 36 | BC9-298 | 23.87 | 24.12 | 24.00 | 2727.0 | 2320.1 | 2523.5 | 50 | Pos | P2, P3 | Aug. 25, 1999 |
| 37 | BC9-540 | 22.10 | 22.01 | 22.06 | 8614.0 | 9091.2 | 8852.6 | 50 | Pos | P1 | Aug. 25, 1999 |

*indicates that the sample's internal control was weakly amplified (W Amp), or very weakly amplified (V W Amp).
Cut-off for positive is 10 pg.

TABLE 5
BCR-ABL Validation Studies Normal

| | | | Ct | | | Quantity (pg) | | | cDNA | | | |
|---|---|---|---|---|---|---|---|---|---|---|---|---|
| | Sample | | #1 | #2 | Ave. | #1 | #2 | Average | (ng) | TaqMan | | Ass Date |
| 1 | 14-1 | (TRlzol) | 45.00 | 45.00 | 45.00 | 0.0 | 0.0 | 0.0 | 100 | Not Detec | Normal | Aug. 10, 1999 |
| 2 | 14-1 | (RNA S | 39.61 | 45.00 | 42.31 | 0.1 | 0.0 | 0.0 | 100 | Not Detec | Normal | Aug. 10, 1999 |
| 3 | 10-1 | | 45.00 | 45.00 | 45.00 | 0.0 | 0.0 | 0.0 | 100 | Not Detec | Normal | Aug. 10, 1999 |
| 4 | 33-1 | | 45.00 | 45.00 | 45.00 | 0.0 | 0.0 | 0.0 | 100 | Not Detec | Normal | Aug. 10, 1999 |
| 5 | 147-1 | | 45.00 | 45.00 | 45.00 | 0.0 | 0.0 | 0.0 | 100 | Not Detec | Normal | Aug. 10, 1999 |
| 6 | 199-1 | | 41.58 | 45.00 | 43.29 | 0.0 | 0.0 | 0.0 | 100 | Not Detec | Normal | Aug. 10, 1999 |
| 7 | 250-1 | | 36.65 | 45.00 | 40.83 | 0.3 | 0.0 | 0.2 | 100 | Not Detec | Normal | Aug. 10, 1999 |
| 8 | 448-1 | | 45.00 | 35.51 | 40.26 | 0.0 | 0.7 | 0.3 | 100 | Not Detec | Normal | Aug. 10, 1999 |
| 9 | 607-1 | | 45.00 | 45.00 | 45.00 | 0.0 | 0.0 | 0.0 | 100 | Not Detec | Normal | Aug. 10, 1999 |
| 10 | 608-1 | | 45.00 | 45.00 | 45.00 | 0.0 | 0.0 | 0.0 | 100 | Not Detec | Normal | Aug. 10, 1999 |
| 11 | 159-1 | | 45.00 | 45.00 | 45.00 | 0.0 | 0.0 | 0.0 | 100 | Not Detec | Normal | Aug. 11, 1999 |
| 12 | 348-1 | | 45.00 | 36.20 | 40.60 | 0.0 | 0.3 | 0.2 | 100 | Not Detec | Normal | Aug. 11, 1999 |
| 13 | 384-1 | | 45.00 | 45.00 | 45.00 | 0.0 | 0.0 | 0.0 | 100 | Not Detec | Normal | Aug. 11, 1999 |
| 14 | 394-1 | | 45.00 | 45.00 | 45.00 | 0.0 | 0.0 | 0.0 | 100 | Not Detec | Normal | Aug. 11, 1999 |
| 15 | 410-1 | | 45.00 | 45.00 | 45.00 | 0.3 | 0.0 | 0.2 | 100 | Not Detec | Normal | Aug. 11, 1999 |
| 16 | 432-1 | | 45.00 | 37.30 | 41.15 | 0.0 | 0.2 | 0.1 | 100 | Not Detec | Normal | Aug. 11, 1999 |
| 17 | 445-1 | | 45.00 | 42.69 | 43.85 | 0.0 | 0.0 | 0.0 | 100 | Not Detec | Normal | Aug. 11, 1999 |
| 18 | 610-1 | | 45.00 | 45.00 | 45.00 | 0.0 | 0.0 | 0.0 | 100 | Not Detec | Normal | Aug. 11, 1999 |
| 19 | 86-1 | | 42.98 | 45.00 | 43.99 | 0.0 | 0.0 | 0.0 | 100 | Not Detec | Normal | Aug. 12, 1999 |
| 20 | 105-1 | | 45.00 | 45.00 | 45.00 | 0.0 | 0.0 | 0.0 | 100 | Not Detec | Normal | Aug. 12, 1999 |
| 21 | 194-1 | | 29.67 | 42.95 | 36.31 | 34.0 | 0.0 | 17.0 | 100 | ? | Normal | Aug. 12, 1999 |
| 22 | 285-1 | | 45.00 | 45.00 | 45.00 | 0.0 | 0.0 | 0.0 | 100 | Not Detec | Normal | Aug. 12, 1999 |
| 23 | 294-1 | | 45.00 | 43.34 | 44.17 | 0.0 | 0.0 | 0.0 | 100 | Not Detec | Normal | Aug. 12, 1999 |
| 24 | 594-1 | | 45.00 | 45.00 | 45.00 | 0.0 | 0.0 | 0.0 | 100 | Not Detec | Normal | Aug. 12, 1999 |
| 25 | 194-1 | | 45.00 | 36.71 | 40.86 | 0.0 | 0.8 | 0.4 | 50 | Not Detec | Normal | Aug. 23, 1999 |

What is claimed is:

1. A method for determining bcr-abl translocation rearrangements in a biological sample comprising the steps of:
a) extracting RNA from a biological sample;
b) quantifying the extracted RNA;
c) reverse transcribing the RNA to cDNA;
d) amplifying the cDNA and detecting a cDNA signal by using the primers and probes set forth in SEQ. ID. NOS. 1-8;
e) obtaining a standard curve of cDNA signals from serial dilutions of a leukemic cell line, wherein the cDNA is obtained by repeating steps a)-d) with the RNA from the leukemic cell line and not the sample; and
f) extrapolating a measurement of the leukemic cells present in the sample by comparing the signal from step d) with that from step e).

2. The method of claim 1 wherein the amplification and detection of the cDNA in step d) is accomplished by Real Time PCR.

3. The method of claim 1 wherein the amplification and detection of the cDNA in step e) is accomplished by Real Time PCR.

4. The method of claim 1 wherein the measurement in step f) comprises a measurement of the number of leukemic cells present in the sample, wherein a result of less than a certain number of cells is reported as negative, and a result of more than a certain number is reported as positive.

5. The method of claim 4 wherein wherein a result of less than one leukemic cell per ten thousand total cells is reported as negative, and a result of more than one leukemic cell per ten thousand total cells is reported as positive.

6. The method of claim 1 wherein the measurement in step f) comprises a measurement of the total number of leukemic cells present in the sample.

7. The method of claim 1 further comprising the step of running the cDNA PCR products of step d) on an electrophoretic gel to obtain fragment size and hence identity information.

8. The method of claim 1 wherein the amplification and detection of the cDNA in step d) is performed in a single container.

9. The method of claim 1 wherein the amplification and detection of the cDNA in step e) is performed in a single container.

10. A method of diagnosing CML or ALL by performing the assay of claim 1.

11. A method of diagnosing CML or ALL by performing the assay of claim 7.

12. The method of claim 1 wherein the sample includes an RNAse inhibitor.
